(19) United States
(12) Patent Application Publication
(10) Pub. No.: US 2023/0340398 A1
(43) Pub. Date: Oct. 26, 2023

(54) METHODS FOR LYSING BACTERIAL CELLS

(71) Applicants: Cellular Biomedicine Group, Inc., Rockville, MD (US); CELLULAR BIOMEDICINE GROUP HK LIMITED, Hong Kong (CN); SHANGHAI CELLULAR BIOPHARMACEUTICAL GROUP LTD., Shanghai (CN); WUXI CELLULAR BIOPHARMACEUTICAL GROUP LTD., Wuxi, Jiangsu (CN)

(72) Inventors: Weidi ZHANG, Shanghai (CN); Jixing FU, Shanghai (CN); Yina CHANG, Shanghai (CN); Yi HONG, Shanghai (CN); Li ZHANG, Shanghai (CN)

(21) Appl. No.: 18/044,263

(22) PCT Filed: Sep. 9, 2021
(86) PCT No.: PCT/US21/49633

§ 371 Date: Mar. 7, 2023

(30) Foreign Application Priority Data
Sep. 10, 2020 (CN) 2020109496845

(51) Int. Cl. C12N 1/06; C12N 15/10

(52) U.S. Cl. CPC C12N 1/06; C12N 15/1003; C12R 2001/19

(57) ABSTRACT

The present disclosure provides methods for lysing bacterial cells. A lysis solution is mixed with a bacterial cell suspension while being shaken. A buffer is then added, and the mixture is shaken. Also provided are methods for isolating plasmids from a bacterial cell lysate.

### CROSS REFERENCE TO RELATED APPLICATION

The present application claims priority to Chinese patent application No. 2020109496845, filed Sep. 10, 2020, which is hereby incorporated by reference in its entirety.

### TECHNICAL FIELD

The present disclosure relates to the field of biotechnology, in particular to methods for lysing bacterial cells.

### BACKGROUND

Gene therapy refers to the introduction of an exogenous therapeutic gene into target cells to correct or compensate for a disease caused by a gene defect or abnormality. Alternatively, a product expressed by the exogenous gene can act on a therapeutic target for treatment.

Plasmids are very useful as vectors in gene therapy. They can be used directly for in vivo treatments, packaged into viral vectors, or used to modify target cells (such as T cells or stem cells) in vitro which can then be administered to patients for treatment.

The purity of plasmids (including supercoil content and endotoxin levels, etc.) can affect the packaging of viral vectors and the efficacy of gene therapy. There are different types of plasmids including open circular plasmids, supercoiled plasmids, linear plasmids, replication intermediates, and so on. The open circular plasmids may result in the inactivation of certain functional genes, which can reduce the efficiency of virus packaging or the efficacy in vivo. Therefore, the supercoil content is a key quality attribute of plasmids, and also a key control point in the purification process of plasmids. Although a purification process can remove part of the open circular plasmids, minimizing the formation of open circular plasmids is key to plasmid production.

Engineered plasmids can be produced by fermentation of Escherichia coli (E. coli). Alkaline lysis is the common method for extracting plasmids from E. coli. Open circular plasmids are mostly produced in the process of bacterial lysis. The traditional process uses manual lysis methods or stirring lysis methods. The efficiency of the former methods varies from operator to operator. It is not easy to control, and the methods are generally not compatible with high throughput. The latter methods may apply too much shear force, which may cause great damage to plasmids, especially large plasmids. This can easily lead to the formation of open circular plasmids.

Therefore, there is a need to develop a simple, convenient and highly efficient method for plasmid purification.

### SUMMARY

One object of the present disclosure is to provide a method for lysing bacterial cells. The present bacterial cell lysis method can significantly improve the mixing of bacterial cells and a lysis solution, so that the plasmids can be released from the bacterial cells, and the yield and quality of the plasmids are improved.

In a first aspect of the present disclosure, a method for lysing bacterial cells is provided. The method may comprise the following steps:
(a) adding a lysis solution to a bacterial cell suspension to obtain a first mixture, shaking the first mixture at a rotation speed ranging from about 10 rpm to about 30 rpm, or from about 10 rpm to about 20 rpm, for about 1 minute to about 10 minutes, about 1 minute to about 8 minutes, or about 3 minutes to about 8 minutes, and incubating the first mixture to obtain a preliminary bacterial cell lysate; and
(b) adding a buffer to the preliminary bacterial cell lysate to obtain a second mixture, shaking the second mixture at a rotation speed ranging from about 10 rpm to about 70 rpm, from about 30 rpm to about 55 rpm, or from about 40 rpm to about 50 rpm, for about 1 minute to about 10 minutes, or about 3 minutes to about 8 minutes, and incubating the second mixture to obtain a bacterial cell lysate.

In certain embodiments, the lysis solution is an alkaline lysis buffer.

In one embodiment, the lysis solution comprises NaOH and/or SDS.

In certain embodiments, the lysis solution comprises NaOH having a concentration ranging from about 50 mM to about 400 mM, from about 100 mM to about 400 mM, from about 100 mM to about 300 mM, from about 100 mM to about 200 mM, from about 200 mM to about 400 mM, from about 200 mM to about 300 mM, from about 50 mM to about 300 mM, from about 50 mM to about 200 mM, from about 5 mM to about 100 mM, from about 150 mM to about 250 mM, from about 150 mM to about 200 mM, from about 200 mM to about 250 mM, about 50 mM, about 100 mM, about 150 mM, about 200 mM, about 250 mM, or about 300 mM.

In certain embodiments, the lysis solution comprises SDS having a concentration ranging from about 0.5% (w/v) to about 2% (w/v), from about 0.5% (w/v) to about 1.5% (w/v), from about 0.5% (w/v) to about 1% (w/v), from about 1% (w/v) to about 2% (w/v), from about 1% (w/v) to about 1.5% (w/v), from about 1.5% (w/v) to about 2% (w/v), about 0.5% (w/v), about 1% (w/v), about 1.5% (w/v), or about 2% (w/v).

In one embodiment, in step (a), the lysis solution is added to the bacterial cell suspension at a flow rate ranging from about 50 ml/min to about 400 ml/min, from about 50 ml/min to about 300 ml/min, from about 50 ml/min to about 200 ml/min, or from about 150 ml/min to about 200 ml/min, while the bacterial cell suspension is being shaken.

In one embodiment, in step (a), the first mixture is incubated for about 1 minute to about 60 minutes, about 1 minute to about 8 minutes, or about 1 minute to about 5 minutes.

In one embodiment, in step (b), the second mixture is incubated for about 1 minute to about 60 minutes, about 1 minute to about 8 minutes, or about 3 minutes to about 8 minutes.

In one embodiment, in step (b), the buffer comprises an acetate buffer (such as potassium acetate buffer).

In one embodiment, in step (b), the buffer is added to the preliminary bacterial cell lysate at a flow rate ranging from about 50 ml/min to about 400 ml/min, from about 50 ml/min to about 300 ml/min, from about 50 ml/min to about 200 ml/min, or from about 150 ml/min to about 200 ml/min, while the preliminary bacterial cell lysate is being shaken.

In one embodiment, the bacterial cell suspension includes a buffer.

In one embodiment, the bacterial cell suspension is prepared by suspending bacterial cells in a suspension buffer, and shaking the suspended bacterial cells to obtain the bacterial cell suspension.

In one embodiment, the suspension buffer comprises a Tris buffer.

In one embodiment, the suspended bacterial cells are shaken at a rotation speed ranging from about 10 rpm to about 70 rpm, or from about 10 rpm to about 20 rpm.

In one embodiment, the suspended bacterial cells are shaken for about 1 minute to about 5 minutes.

In certain embodiments, a first shaker is used in step (a), a second shaker is used in step (b), and a third shaker is used when preparing the bacterial cell suspension.

In one embodiment, the first shaker, the second shaker, and the third shaker are the same shaker or are different shakers.

In one embodiment, the first shaker is a 3D or 2D shaker; and/or the second shaker is a 3D or 2D shaker.

In one embodiment, the third shaker is a 3D or 2D shaker.

In one embodiment, the first shaker is a back and forth shaking shaker, a left and right shaking shaker, and/or a wave shaker.

In one embodiment, the second shaker is a back and forth shaking shaker, a left and right shaking shaker, and/or a wave shaker.

In one embodiment, the third shaker is a back and forth shaking shaker, a left and right shaking shaker, and/or a wave shaker.

In one embodiment, the first shaker includes a first tray.

In one embodiment, the second shaker includes a second tray.

In one embodiment, the third shaker includes a third tray.

In one embodiment, in step (a), the first mixture is placed on a first tray for shaking, where the first tray forms an angle of about 10 to about 20 degrees with respect to a horizontal plane.

In one embodiment, the bacterial cell suspension is placed in a liquid dispensing bag.

In one embodiment, in step (b), the preliminary bacterial cell lysate is placed on a second tray for shaking, where the second tray forms an angle of about 10 to about 20 degrees with respect to a horizontal plane.

In one embodiment, the preliminary bacterial cell lysate is placed in a liquid dispensing bag.

In one embodiment, the bacterial cells are engineered bacterial cells comprising at least one plasmid.

In one embodiment, the plasmid is P1 plasmid.

In one embodiment, the bacterial cells are Top10 Escherichia coli.

In a second aspect of the present disclosure, a method for preparing a plasmid is provided. The method may comprise the following steps:
(a) lysing bacterial cells using the present method for lysing bacterial cells to obtain a bacterial cell lysate, wherein the bacterial cells comprising the plasmid; and
(b) isolating the plasmid from the bacterial cell lysate.

It should be understood that within the scope of the present disclosure, the above-mentioned technical features of the present disclosure and the technical features specifically described in the following (such as the embodiments) can be combined with each other to form a new or preferred technical solution. Due to space limitations, this will not be repeated herein.

### BRIEF DESCRIPTION OF THE DRAWINGS

FIG. 1 shows the result of DNA gel electrophoresis of the lysis products.

FIG. 2 shows the bands of the lysis products in the box shown in lane 2 in the DNA gel electrophoresis result of FIG. 1.

FIG. 3 shows the bands of the lysis products in the box shown in lane 3 in the DNA gel electrophoresis result of FIG. 1.

### DETAILED DESCRIPTION

The present disclosure provides for a method for bacterial cell lysis. Compared with the traditional manual lysis method or the stirring lysis method, the bacterial cell lysis method of the present disclosure can significantly improve the sufficient mixing of the bacterial cells and the lysis solution, thereby releasing the plasmid from the bacterial cells, increasing the yield of the plasmid, and increasing the concentration and supercoil content of plasmids.

The present disclosure provides for a method for lysing bacterial cells. The method may comprise the following steps:
(a) adding a lysis solution to a bacterial cell suspension to obtain a first mixture, shaking the first mixture at a rotation speed ranging from about 10 rpm to about 30 rpm, from about 10 rpm to about 20 rpm, from about 10 rpm to about 25 rpm, from about 10 rpm to about 15 rpm, from about 15 rpm to about 30 rpm, from about 15 rpm to about 25 rpm, from about 15 rpm to about 20 rpm, from about 20 rpm to about 30 rpm, from about 20 rpm to about 25 rpm, from about 25 rpm to about 30 rpm, about 10 rpm, about 15 rpm, about 20 rpm, about 25 rpm, or about 30 rpm, for about 1 minute to about 10 minutes, about 1 minute to about 5 minutes, about 1 minute to about 8 minutes, about 2 minutes to about 10 minutes, about 2 minutes to about 8 minutes, about 2 minutes to about 5 minutes, about 3 minutes to about 5 minutes, about 3 minutes to about 8 minutes, about 3 minutes, about 4 minutes, about 5 minutes, about 6 minutes, about 7 minutes, or about 8 minutes, and incubating (without shaking) the first mixture to obtain a preliminary bacterial cell lysate; and
(b) adding a buffer to the preliminary bacterial cell lysate to obtain a second mixture, shaking the second mixture at a rotation speed ranging from about 10 rpm to about 70 rpm, from about 30 rpm to about 55 rpm, from about 10 rpm to about 65 rpm, from about 10 rpm to about 60 rpm, from about 10 rpm to about 55 rpm, from about 10 rpm to about 50 rpm, from about 10 rpm to about 45 rpm, from about 10 rpm to about 40 rpm, from about 10 rpm to about 35 rpm, from about 10 rpm to about 30 rpm, from about 15 rpm to about 70 rpm, from about 15 rpm to about 65 rpm, from about 15 rpm to about 60 rpm, from about 15 rpm to about 55 rpm, from about 15 rpm to about 50 rpm, from about 15 rpm to about 45 rpm, from about 15 rpm to about 40 rpm, from about 15 rpm to about 35 rpm, from about 15 rpm to about 30 rpm, from about 20 rpm to about 70 rpm, from about 20 rpm to about 65 rpm, from about 20 rpm to about 60 rpm, from about 20 rpm to about 55 rpm, from about 20 rpm to about 50 rpm, from about 20 rpm to about 45 rpm, from about 20 rpm to about 40 rpm, from about 20 rpm to about 35 rpm, from about 20 rpm to about 30 rpm, from about 25 rpm to about 70 rpm, from about 25 rpm to about 65 rpm, from about 25 rpm to about 60 rpm, from about 25 rpm to about 55 rpm, from about 25 rpm to about 50 rpm, from about 25 rpm to about 45 rpm, from about 25 rpm to about 40 rpm, from about 25 rpm to about 35 rpm, from about 30 rpm to about 70 rpm, from about 30 rpm to about 65 rpm, from about 30 rpm to about 60 rpm, from about 30 rpm to about 55 rpm, from about 30 rpm to about 50 rpm, from about 30 rpm to about 45 rpm, from about 30 rpm to about 40 rpm, from about 35 rpm to about 70 rpm, from about 35 rpm to about 65 rpm, from about 35 rpm to about 60 rpm, from about 35 rpm to about 55 rpm, from about 35 rpm to about 50 rpm, from about 35 rpm to about 45 rpm, from about 35 rpm to about 40 rpm, from about 40 rpm to about 70 rpm, from about 40 rpm to about 65 rpm, from about 40 rpm to about 60 rpm, from about 40 rpm to about 55 rpm, from about 40 rpm to about 50 rpm, from about 40 rpm to about 45 rpm, from about 45 rpm to about 70 rpm, from about 45 rpm to about 65 rpm, from about 45 rpm to about 60 rpm, from about 45 rpm to about 55 rpm, from about 45 rpm to about 50 rpm, about 30 rpm, about 35 rpm, about 40 rpm, about 45 rpm, about 50 rpm, about 55 rpm, or about 60 rpm, for about 1 minute to about 10 minutes, about 1 minute to about 5 minutes, about 1 minute to about 8 minutes, about 2 minutes to about 10 minutes, about 2 minutes to about 8 minutes, about 2 minutes to about 5 minutes, about 3 minutes to about 5 minutes, about 3 minutes to about 8 minutes, about 3 minutes, about 4 minutes, about 5 minutes, about 6 minutes, about 7 minutes, or about 8 minutes, and incubating (without shaking) the second mixture to obtain a bacterial cell lysate.

In certain embodiments, the lysis solution comprises one or more hydroxides. The hydroxide may be a hydroxide of an alkali metal, such as sodium hydroxide (NaOH), potassium hydroxide (KOH), lithium hydroxide (LiOH), etc. The hydroxide may be a hydroxide of an alkaline earth metal, such as magnesium hydroxide (Mg(OH)₂), calcium hydroxide (Ca(OH)₂), etc.

In certain embodiments, the lysis solution comprises one or more surfactants or detergents. The surfactant may be an anionic surfactant, such as sodium dodecyl sulfate (SDS).

In one embodiment, in step (a), the lysis solution is added to the bacterial cell suspension at a flow rate ranging from about 50 ml/min to about 400 ml/min, from about 50 ml/min to about 350 ml/min, from about 50 ml/min to about 300 ml/min, from about 50 ml/min to about 250 ml/min, from about 50 ml/min to about 200 ml/min, from about 100 ml/min to about 400 ml/min, from about 100 ml/min to about 350 ml/min, from about 100 ml/min to about 300 ml/min, from about 100 ml/min to about 250 ml/min, from about 100 ml/min to about 200 ml/min, from about 150 ml/min to about 400 ml/min, from about 150 ml/min to about 350 ml/min, from about 150 ml/min to about 300 ml/min, from about 150 ml/min to about 250 ml/min, from about 150 ml/min to about 200 ml/min, from about 200 ml/min to about 400 ml/min, from about 200 ml/min to about 350 ml/min, from about 200 ml/min to about 300 ml/min, from about 200 ml/min to about 250 ml/min, about 150 ml/min, about 200 ml/min, or about 250 ml/min, while the bacterial cell suspension is being shaken.

In certain embodiments, in step (a), the volume ratio of the lysis solution to the bacterial cell suspension ranges from about 1:4 to about 1:1, from about 1:3 to about 1:1, from about 1:2 to about 1:1, about 1:4, about 1:3.5, about 1:3, about 1:2.5, about 1:2, about 1:1.5, or about 1:1.

In one embodiment, in step (a), the first mixture is incubated for about 1 minute to about 60 minutes, about 1 minute to about 45 minutes, for about 1 minute to about 30 minutes, about 1 minute to about 20 minutes, about 1 minute to about 15 minutes, about 1 minute to about 10 minutes, about 1 minute to about 8 minutes, about 1 minute to about 5 minutes, about 2 minutes to about 45 minutes, about 2 minutes to about 30 minutes, about 2 minutes to about 20 minutes, about 2 minutes to about 15 minutes, about 2 minutes to about 10 minutes, about 2 minutes to about 8 minutes, about 2 minutes to about 5 minutes, about 3 minutes to about 5 minutes, about 3 minutes to about 8 minutes, about 5 minutes to about 45 minutes, about 5 minutes to about 30 minutes, about 5 minutes to about 20 minutes, about 5 minutes to about 15 minutes, about 5 minutes to about 10 minutes, about 2 minutes, about 3 minutes, about 4 minutes, about 5 minutes, about 6 minutes, about 7 minutes, or about 8 minutes.

In certain embodiments, in step (a), the first mixture is shaken and/or incubated at a temperature ranging from about 20° C. to about 25° C., or at an ambient temperature. Hydrolysis may be conducted at a temperature ranging from about 1° C. to about 37° C., from about 2° C. to about 37° C., from about 10° C. to about 15° C., from about 15° C. to about 20° C., from about 20° C. to about 25° C., from about 25° C. to about 30° C., from about 30° C. to about 35° C., from about 4° C. to about 35° C., from about 10° C. to about 37° C., from about 15° C. to about 37° C., from about 20° C. to about 37° C., from about 25° C. to about 37° C., from about 2° C. to about 8° C., from about 4° C. to about 8° C., from about 2° C. to about 4° C., from about 6° C. to about 8° C., from about 4° C. to about 10° C., from about 4° C. to about 15° C., from about 4° C. to about 20° C., from about 4° C. to about 25° C., from about 4° C. to about 30° C., from about 4° C. to about 35° C., from about 4° C. to about 37° C., about 4° C., about 25° C., or about 37° C. The temperature may be in a range between any two integer value temperatures selected from about 1° C. to about 37° C. The temperature may be any one integer value temperature selected from those including and between about 1° C. and about 37° C., or between about 15° C. and about 35° C. Temperatures between room temperature (ambient temperature) and about 37° C. may be used. The temperature may be any one temperature including and between room temperature and about 37° C. Temperatures between about 20° C. and about 35° C. may be used. The temperature may be any temperature including and between about 20° C. and about 25° C. The temperature may be about 25° C.

In certain embodiments, in step (b), the volume ratio of the buffer to the preliminary bacterial cell lysate ranges from about 1:4 to about 1:1, from about 1:3 to about 1:1, from about 1:2 to about 1:1, about 1:4, about 1:3.5, about 1:3, about 1:2.5, about 1:2, about 1:1.5, or about 1:1.

In one embodiment, in step (b), the second mixture is incubated for about 1 minute to about 60 minutes, about 1 minute to about 45 minutes, for about 1 minute to about 30 minutes, about 1 minute to about 20 minutes, about 1 minute to about 15 minutes, about 1 minute to about minutes, about 1 minute to about 8 minutes, about 1 minute to about 5 minutes, about 2 minutes to about 45 minutes, about 2 minutes to about 30 minutes, about 2 minutes to about 20 minutes, about 2 minutes to about 15 minutes, about 2 minutes to about 10 minutes, about 2 minutes to about 8 minutes, about 2 minutes to about 5 minutes, about 3 minutes to about 5 minutes, about 3 minutes to about 8 minutes, about 5 minutes to about 45 minutes, about 5 minutes to about 30 minutes, about 5 minutes to about 20 minutes, about 5 minutes to about 15 minutes, about 5 minutes to about 10 minutes, about 2 minutes, about 3 minutes, about 4 minutes, about 5 minutes, about 6 minutes, about 7 minutes, about 8 minutes, about 9 minutes, about 10 minutes.

In certain embodiments, in step (b), the second mixture is shaken and/or incubated at a temperature ranging from about 20° C. to about 25° C., or at an ambient temperature. Hydrolysis may be conducted at a temperature ranging from about 1° C. to about 37° C., from about 2° C. to about 37° C., from about 10° C. to about 15° C., from about 15° C. to about 20° C., from about 20° C. to about 25° C., from about 25° C. to about 30° C., from about 30° C. to about 35° C., from about 4° C. to about 35° C., from about 10° C. to about 37° C., from about 15° C. to about 37° C., from about 20° C. to about 37° C., from about 25° C. to about 37° C., from about 2° C. to about 8° C., from about 4° C. to about 8° C., from about 2° C. to about 4° C., from about 6° C. to about 8° C., from about 4° C. to about 10° C., from about 4° C. to about 15° C., from about 4° C. to about 20° C., from about 4° C. to about 25° C., from about 4° C. to about 30° C., from about 4° C. to about 35° C., from about 4° C. to about 37° C., about 4° C., about 25° C., or about 37° C. The temperature may be in a range between any two integer value temperatures selected from about 1° C. to about 37° C. The temperature may be any one integer value temperature selected from those including and between about 1° C. and about 37° C., or between about 15° C. and about 35° C. Temperatures between room temperature (ambient temperature) and about 37° C. may be used. The temperature may be any one temperature including and between room temperature and about 37° C. Temperatures between about 20° C. and about 35° C. may be used. The temperature may be any temperature including and between about 20° C. and about 25° C. The temperature may be about 25° C.

The buffer may be an acetate buffer or a citrate buffer. The acetate buffer may be potassium acetate buffer and/or sodium acetate buffer. In certain embodiments, the buffer is potassium acetate buffer having a concentration ranging from about 1 M (mol/L) to about 5 M, from about 1 M to about 4 M, from about 1 M to about 3 M, from about 1 M to about 2 M, from about 2 M to about 5 M, from about 2 M to about 4 M, from about 2 M to about 3 M, from about 3 M to about 5 M, from about 3 M to about 4 M, about 1 M, about 1.5 M, about 2 M, about 2.5 M, about 3 M, about 3.5 M, about 4 M, about 4.5 M, or about 5 M.

The buffer may have a pH ranging from about pH 3 to about pH 7, from about pH 3 to about pH 6.5, from about pH 3 to about pH 6, from about pH 3 to about pH 5.5, from about pH 3 to about pH 5, from about pH 3 to about pH 4, from about pH 4 to about pH 7, from about pH 4 to about pH 6.5, from about pH 4 to about pH 6, from about pH 4 to about pH 5.5, from about pH 4 to about pH 5, from about pH 5 to about pH 7, from about pH 5 to about pH 6.5, from about pH 5 to about pH 6, from about pH 5 to about pH 5.5, from about pH 5.5 to about pH 6, about pH 4 about pH 4.5, about pH 5, about pH 5.5, or about pH 6.

In one embodiment, in step (b), the buffer comprises an acetate buffer (such as potassium acetate buffer).

In one embodiment, in step (b), the buffer is added to the preliminary bacterial cell lysate at a flow rate ranging from about 50 ml/min to about 400 ml/min, from about 50 ml/min to about 350 ml/min, from about 50 ml/min to about 300 ml/min, from about 50 ml/min to about 250 ml/min, from about 50 ml/min to about 200 ml/min, from about 100 ml/min to about 400 ml/min, from about 100 ml/min to about 350 ml/min, from about 100 ml/min to about 300 ml/min, from about 100 ml/min to about 250 ml/min, from about 100 ml/min to about 200 ml/min, from about 150 ml/min to about 400 ml/min, from about 150 ml/min to about 350 ml/min, from about 150 ml/min to about 300 ml/min, from about 150 ml/min to about 250 ml/min, from about 150 ml/min to about 200 ml/min, from about 200 ml/min to about 400 ml/min, from about 200 ml/min to about 350 ml/min, from about 200 ml/min to about 300 ml/min, from about 200 ml/min to about 250 ml/min, about 150 ml/min, about 200 ml/min, or about 250 ml/min, while the preliminary bacterial cell lysate is being shaken.

In one embodiment, the bacterial cell suspension includes a buffer.

In one embodiment, the bacterial cell suspension is prepared by suspending bacterial cells in a suspension buffer, and shaking the suspended bacterial cells to obtain the bacterial cell suspension.

The suspension buffer may be a Tri buffer, a TAPS buffer, a Bicine buffer, a Tricine buffer, a TAPSO buffer, a HEPES buffer, a TES buffer, a MOPS buffer, or a PIPES buffer.

The suspension buffer may have a pH ranging from about pH 6 to about pH 9, from about pH 6.5 to about pH 9, from about pH 7 to about pH 9, from about pH 7.5 to about pH 9, from about pH 8 to about pH 9, from about pH 8.5 to about pH 9, from about pH 6 to about pH 8, from about pH 6.5 to about pH 8, from about pH 7 to about pH 8, from about pH 7.5 to about pH 8, from about pH 6 to about pH 8.5, from about pH 7 to about pH 8.5, from about pH 8 to about pH 8.5, about pH 7, about pH 7.5, about pH 8, about pH 8.5, or about pH 9.

In one embodiment, the suspension buffer comprises a Tris buffer.

In one embodiment, the suspended bacterial cells are shaken at a rotation speed ranging from about 10 rpm to about 30 rpm, from about 10 rpm to about 20 rpm, from about 10 rpm to about 25 rpm, from about 10 rpm to about 15 rpm, from about 15 rpm to about 30 rpm, from about 15 rpm to about 25 rpm, from about 15 rpm to about 20 rpm, from about 20 rpm to about 30 rpm, from about 20 rpm to about 25 rpm, from about 25 rpm to about 30 rpm, about 10 rpm, about 15 rpm, about 20 rpm, about 25 rpm, or about 30 rpm.

In one embodiment, the suspended bacterial cells are shaken for about 1 minute to about 60 minutes, about 1 minute to about 45 minutes, about 1 minute to about 30 minutes, about 1 minute to about 20 minutes, about 1 minute to about 10 minutes, about 1 minute to about 5 minutes, about 1 minute to about 4 minutes, about 1 minute to about 3 minutes, about 1 minute, about 2 minutes, about 3 minutes, about 4 minutes, or about 5 minutes.

In certain embodiments, a first shaker is used in step (a), a second shaker is used in step (b), and a third shaker is used when preparing the bacterial cell suspension.

In one embodiment, the first shaker, the second shaker, and the third shaker are the same shaker or are different shakers.

In one embodiment, the first shaker is a 3D (3-dimensional) or 2D shaker; and/or the second shaker is a 3D or 2D shaker.

In one embodiment, the third shaker is a 3D or 2D shaker.

In one embodiment, the first shaker is a back and forth shaking shaker, a left and right shaking shaker, and/or a wave shaker.

In one embodiment, the second shaker is a back and forth shaking shaker, a left and right shaking shaker, and/or a wave shaker.

In one embodiment, the third shaker is a back and forth shaking shaker, a left and right shaking shaker, and/or a wave shaker.

In one embodiment, the lysis solution comprises a NaOH/SDS lysis solution.

In one embodiment, the lysis solution comprises NaOH and/or SDS.

In one embodiment, the first shaker includes a first tray.

In one embodiment, the second shaker includes a second tray.

In one embodiment, the third shaker includes a third tray.

In one embodiment, in step (a), the first mixture is placed on a first tray for shaking, where the first tray forms an angle of about 10 to about 20 degrees with respect to a horizontal plane.

In one embodiment, the bacterial cell suspension is placed in a liquid dispensing bag.

In one embodiment, in step (b), the preliminary bacterial cell lysate is placed on a second tray for shaking, where the second tray forms an angle of about 10 to about 20 degrees with respect to a horizontal plane.

In one embodiment, the preliminary bacterial cell lysate is placed in a liquid dispensing bag.

In one embodiment, the bacterial cells are engineered bacterial cells comprising at least one plasmid.

In certain embodiments, the bacterial cells are Escherichia coli bacteria.

In one embodiment, the bacterial cells are Top10 Escherichia coli.

In one embodiment, the plasmid is P1 plasmid.

The present disclosure also provides for a method for preparing a plasmid. The method may comprise the following steps:
(a) lysing bacterial cells using the present method for lysing bacterial cells to obtain a bacterial cell lysate, wherein the bacterial cells comprising the plasmid; and
(b) isolating the plasmid from the bacterial cell lysate.

The bacterial cells can be any suitable bacterial cells. The plasmids can be any suitable types of plasmids.

The present invention provides a method for bacterial cell lysis, and the method includes the following steps:
(1) placing a bacterial cell liquid on a first shaker for shaking, and then adding a lysis solution to the bacterial liquid with shaking, shaking and mixing, and then letting stand to obtain a primary bacterial cell lysate, wherein a rotation speed of the first shaker is 10 to 30 rpm, and the mixing time is 1 to 10 min;
(2) placing the primary bacterial cell lysate on a second shaker for shaking, and then adding a buffer to the primary bacterial cell lysate with shaking, shaking and mixing, and then letting stand to obtain a bacterial cell lysate, wherein a rotation speed of the second shaker is 10 to 70 rpm, and the mixing time is 1 to 10 min.

The bacterial cells may be engineered bacterial cells modified by a plasmid.

The plasmid may include P1 plasmid.

The bacterial cells may include Top10 Escherichia coli engineered bacteria.

In one embodiment of the present disclosure, in the step (1), the rotation speed of the first shaker is 10 to 20 rpm.

In one embodiment of the present disclosure, in the step (1), the standing time is 1 to 60 min, 1 to 8 min, or 1 to 5 min.

In one embodiment of the present disclosure, in the step (1), the mixing time is 1 to 8 min, or 3 to 8 min.

In one embodiment of the present disclosure, in the step (1), the bacterial cell liquid includes a bacterial cell buffer.

Typically, in the step (1), the bacterial cell liquid is prepared by the following method: (1-1) after mixing the bacterial cells with a buffer, shaking and mixing on a third shaker to obtain the bacterial cell liquid.

In one embodiment, in the step (1-1), the buffer includes a Tris buffer.

In one embodiment, in the step (1-1), the rotation speed of the third shaker is 10 to 70 rpm, or 10 to 20 rpm.

In one embodiment, in the step (1-1), the mixing time is 1 to 5 min.

In one embodiment of the present disclosure, in the step (1), the lysis solution is added into the primary bacterial cell lysate with shaking at a flow rate of 50 to 400 ml/min, 50 to 300 ml/min, 50 to 200 ml/min, or 150 to 200 ml/min.

In the method of the present disclosure, the first shaker, the second shaker, and the second shaker are the same shaker or different shakers.

Typically, the first shaker is a 3D or 2D shaker.

Typically, the second shaker is a 3D or 2D shaker.

Typically, the third shaker is a 3D or 2D shaker.

Typically, the first shaker is a front and back shaking shaker, a left and right shaking shaker, and/or a wave shaker.

Typically, the second shaker is a front and back shaking shaker, a left and right shaking shaker, and/or a wave shaker.

Typically, the third shaker is a front and back shaking shaker, a left and right shaking shaker, and/or a wave shaker.

In one embodiment of the present disclosure, in the step (1), the lysis solution includes a NaOH/SDS lysis solution.

In one embodiment, in the step (1), the lysis solution includes NaOH/SDS components.

In one embodiment of the present disclosure, the first shaker includes a first tray.

In one embodiment of the present disclosure, the second shaker includes a second tray.

In one embodiment of the present disclosure, the third shaker includes a third tray.

In one embodiment, in the step (1), the bacterial cell liquid is placed on a first tray for shaking.

In one embodiment, the first tray has an angle of 10 to 20 degrees with a horizontal plane.

In one embodiment, the bacterial cell liquid is placed in a liquid dispensing bag.

In one embodiment, in the step (2), the primary bacterial cell lysate is placed on a second tray for shaking.

In one embodiment, the second tray has an angle of 10 to 20 degrees with a horizontal plane.

In one embodiment, the primary bacterial cell lysate is placed in a liquid dispensing bag.

In one embodiment, in the step (2), the standing time is 1 to 60 min.

In one embodiment, in the step (2), the buffer includes an acetate (such as potassium acetate) buffer.

The buffer is added into the primary bacterial cell lysate with shaking at a flow rate of 50 to 400 ml/min.

In one embodiment of the present disclosure, in the step (2), the buffer is added into the primary bacterial cell lysate with shaking at a flow rate of 50 to 400 ml/min, 50 to 300 ml/min, 50 to 200 ml/min, or 150 to 200 ml/min.

In one embodiment of the present disclosure, in the step (2), the rotation speed of the second shaker is 30 to 55 rpm, or 40 to 50 rpm.

In one embodiment of the present disclosure, in the step (2), the mixing time is 3 to 8 min.

In one embodiment of the present disclosure, in the step (2), the standing time is 1 to 60 min, or 3 to 8 min.

The present disclosure also provides a method for preparing a plasmid; the method includes the following steps:
(a) lysing engineered bacterial cells modified by a plasmid with the method for bacterial cell lysis set forth in claim 1 to obtain a bacterial cell lysate;
(b) isolating the plasmid from the bacterial cell lysate.

The present method offers the following major advantages:
1. The bacterial cell lysis method of the present disclosure has simple operation, saves time and manpower, and is convenient and suitable for industrial production.
2. Compared with the traditional manual lysis method or the stirring lysis method, the bacterial cell lysis method of the present disclosure can significantly improve the sufficient mixing of the bacterial cells and the lysis solution, thereby releasing the plasmid from the bacterial cells, increasing the yield of the plasmid, and increasing the concentration and supercoil content of the plasmid.
3. The bacterial cell lysis method of the present disclosure overcomes the influence of human factors and can be stably scaled up.

The present disclosure will be further explained below in conjunction with specific embodiments. It should be understood that these embodiments are only used to illustrate the present disclosure and not to limit the scope of the present disclosure. The experimental methods that have not been indicated for specific conditions in the following examples usually follow the conventional conditions or the conditions recommended by the manufacturers. Unless otherwise stated, the percentages and parts are calculated by weight.

### EXAMPLE 1

### 1. Culturing and Harvesting Bacteria

Reagents and materials: LB medium; Top10 Escherichia coli; 2 L conical flask; kanamycin or ampicillin concentrate; pipette; and 500 ml centrifuge bottle.

Instruments: constant temperature shaker; high-speed centrifuge; AKTA Flux 6; peristaltic pump.

Inoculation and culturing: A Top10 Escherichia coli starter culture (containing P1 plasmids, the size of which is about 2000 bp) was added into LB medium at a volume ratio of 1:1000. Kanamycin or ampicillin concentrate was added at a volume ratio of 1:500 (concentrate : LB medium). The final concentration of kanamycin or ampicillin was 100 ng/m1˜200 ng/m1. The bacteria were grown at 37° C. for 10 to 30 hours with shaking (260 rpm). The bacterial cells were harvested by centrifugation or ultrafiltration.

### 2. Bacterial Cell Lysis

Reagents and materials: Tris buffer (50 mM Tris-Cl, 10 mM EDTA, 50 mM glucose, pH 8.0); NaOH/SDS lysis solution (1% SDS (w/v)/200 mM NaOH); potassium acetate buffer (3.0 M potassium acetate, pH 5.0); wave type liquid dispensing bag; pipettes; and centrifuge tubes.

Instruments: 3D shaker (SCILOGEX) (which can provide wave type shaking); and peristaltic pump.

### 2.1 Adding Tris Buffer:

75 ml of Tris buffer was added to each of the centrifuge bottles containing the bacterial cells harvested from 400-500 mL bacterial culture. The bacterial cells were resuspended and mixed with the Tris buffer. For the manual lysis method, the bacterial cell suspension in one centrifuge bottle was used for manually lysing the bacterial cells.

For the shaker lysis method, a peristaltic pump was used to pump the 1-2 liters bacterial cell suspension into a 5 L liquid dispensing bag. Then the liquid dispensing bag was placed on the tray of a 3D shaker. The bacterial cells in the liquid dispensing bag were then being incubated with shaking at 15 rpm for approximately 3 minutes.

### 2.2 Adding NaOH/SDS Lysis Solution:

Manual lysis method: 75 ml of NaOH/SDS lysis solution was added to the centrifuge bottle selected for the manual lysis method, and the bottle was then gently inverted 3 times for mixing, before being incubated without shaking for 1 minute.

Shaker lysis method: the rotation speed of a 3D shaker was set as 15 rpm (the angle between the tray of the 3D shaker and the horizontal plane was)15°. 1-2 liters of a NaOH/SDS lysis solution was pumped using a peristaltic pump into the 5 L liquid dispensing bag (containing 1-2 liters of the bacterial cell suspension) at a flow rate of 200 ml/min while the liquid dispensing bag was being shaken. After mixing for 5 minutes, the shaker was stopped, and the sample was incubated without shaking for 2 minutes.

### 2.3 Adding Potassium Acetate Buffer:

Manual lysis method: 75 ml of potassium acetate buffer was added into the centrifuge bottle selected for the manual lysis method, and the bottle was then gently inverted 6 times to mix. A sample of 1 ml of the lysis product was taken and added into a 1.5 ml centrifuge tube for subsequent testing.

Shaker lysis method: the rotation speed of a 3D shaker was set as 45 rpm. 1-2 liters of potassium acetate buffer was pumped using a peristaltic pump into the liquid dispensing bag at a flow rate of 200 ml/min while the liquid dispensing bag was being shaken. After mixing for 5 minutes, the shaker was stopped, and the sample was incubated without shaking for 5 minutes. A sample of 1 ml of the lysis product was then taken and added into a 1.5 ml centrifuge tube.

### 3. Experimental Results

### 1. Plasmid Concentration Determination

Instrument: Ultra-sensitive ultraviolet spectrophotometer

The samples of the two groups of lysis products described above were tested for plasmid concentration with an ultra-sensitive UV spectrophotometer. 2 μl was taken each time for detection, and the testing of each group was repeated 3 times. The results are shown in Table 1 below.

TABLE 1
Results of plasmid concentration test of lysis products

| | | Nucleic Acid | | | | | | |
|---|---|---|---|---|---|---|---|---|
| | Sample | concentration | A260 | A280 | | | Sample | |
| No. | ID | (ng/μl) | (Abs) | (Abs) | 260/280 | 260/230 | Type | Factor |
| 1 | 0392- | 683.1 | 13.662 | 6.379 | 2.14 | 0.97 | DNA | 50 |
| | system | | | | | | | |
| 2 | 0392- | 675.8 | 13.516 | 6.258 | 2.16 | 0.99 | DNA | 50 |
| | system | | | | | | | |
| 3 | 0392- | 679.5 | 13.591 | 6.325 | 2.15 | 0.99 | DNA | 50 |
| | system | | | | | | | |
| 4 | 0392- | 599.2 | 11.983 | 5.828 | 2.06 | 0.94 | DNA | 50 |
| | manual | | | | | | | |
| 5 | 0392- | 599.6 | 11.993 | 5.887 | 2.04 | 0.92 | DNA | 50 |
| | manual | | | | | | | |
| 6 | 0392- | 593.4 | 11.867 | 5.754 | 2.06 | 0.93 | DNA | 50 |
| | manual | | | | | | | |

Note:
Nos. 1 to 3 are the shaker lysis method group, and Nos. 4 to 6 are the manual lysis method group.

As can be seen from Table 1, the average plasmid concentration of the lysis product obtained in the shaker lysis method group (“system”) was 679.5 ng/μl, while the average plasmid concentration of the lysis product obtained in the manual lysis method group (“manual”) was 597.4 ng/μl. Thus, the lysis by shaker method allows the bacterial cells to be fully mixed with the lysis solution, as compared to the lysis by manual operation, so that plasmids were released more fully from the bacterial cells and a higher yield of plasmid can be obtained.

### 2. DNA Gel Electrophoresis Detection and Plasmid Supercoil Content Analysis

Instruments: digital display electrophoresis apparatus, and gel imager.

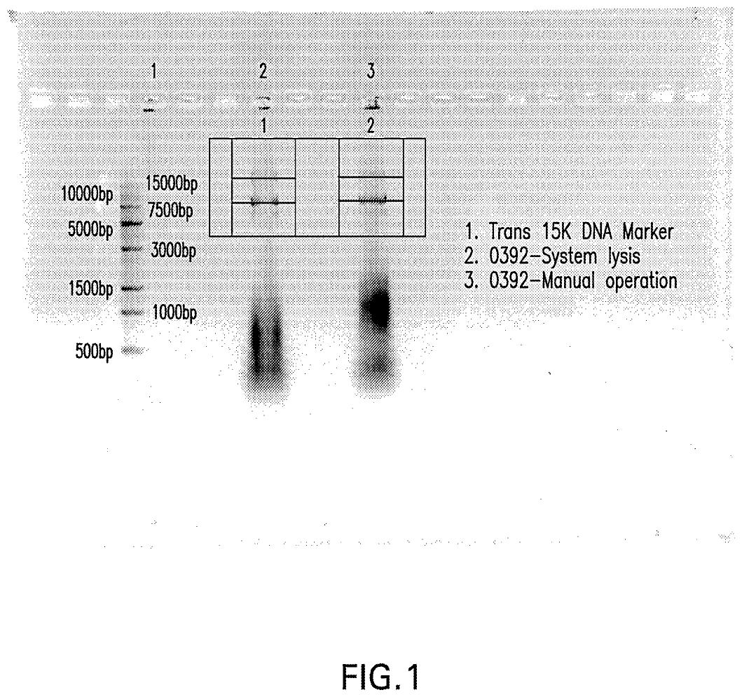

According to the plasmid concentration results, 1000 ng to 1100 ng of the lysis products in each of the two groups were taken for DNA gel electrophoresis detection (120 V, 30 min). After the electrophoresis, a gel imager was used for analysis. The DNA gel electrophoresis results are shown in FIG. 1.

The DNA gel electrophoresis results (FIG. 1) show that the lysis products obtained in the shaker lysis method group (system lysis) and the manual lysis method group contain the plasmid electrophoresis bands at the same positions. This confirms that the plasmids in the bacterial cells did not change due to different lysis methods. The present method obtained the correct lysis products that were consistent with those obtained in the manual method.

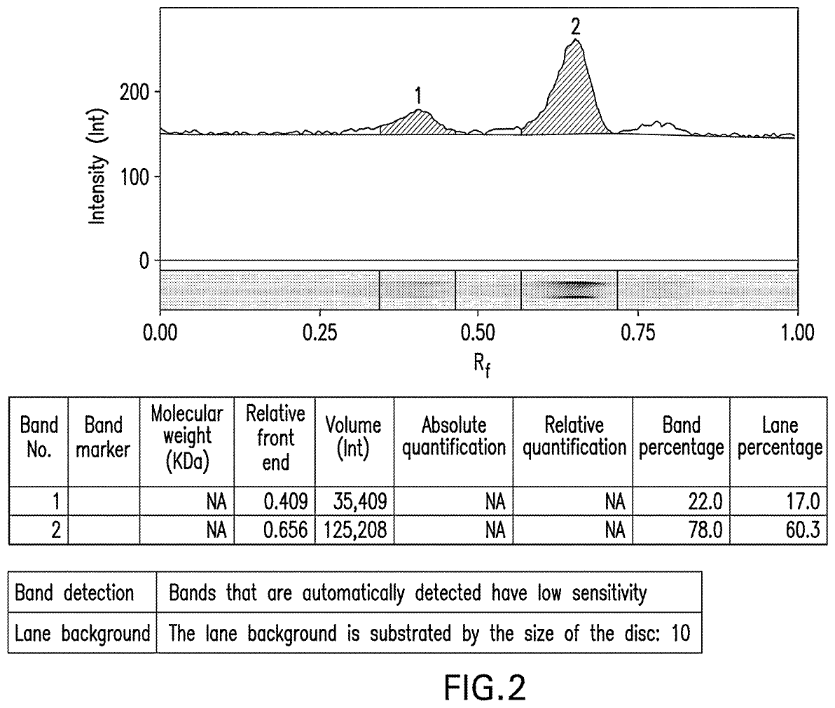

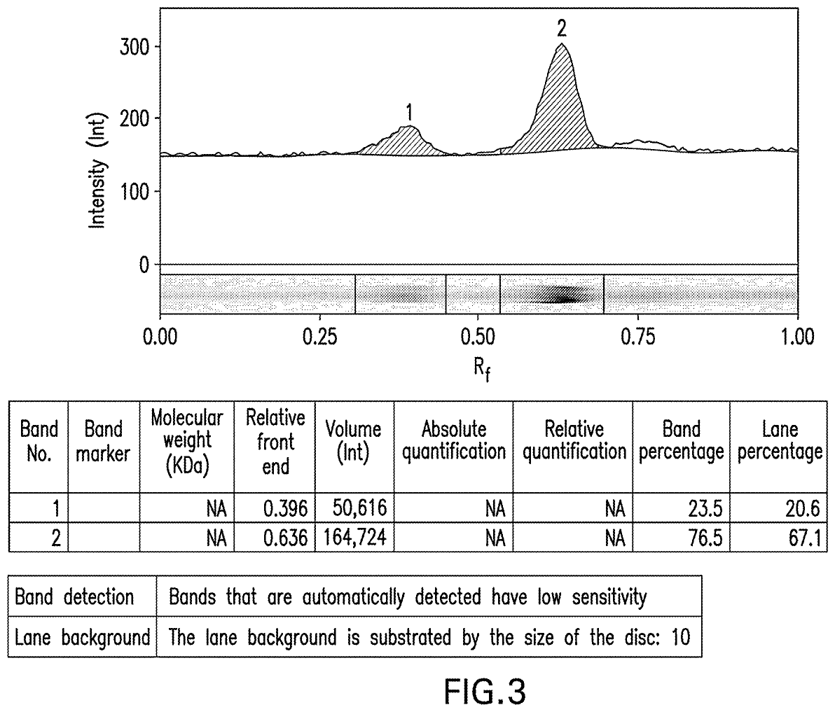

The bands in the DNA gel electrophoresis in FIG. 1 were further analyzed as shown in FIG. 2 and FIG. 3.

An analysis of the supercoil content of the lysis products suggested that the content of supercoiled plasmids in the shaker lysis method group (lane 2) was 78%, while the content of supercoiled plasmids in the manual lysis method group (lane 3) was 76.5%. The shaker operation method allowed the bacterial cells to be fully mixed with the lysis solution. The plasmids isolated using the shaker lysis method had a higher supercoiled content than the manual lysis method group.

### CONCLUSION

When using the same lysis system (e.g., same lysis solution etc.), the lysis method using shakers has several advantages compared with the traditional manual method. The lysis can be controlled and quantified, is efficient, and produces higher concentration of plasmids with higher supercoil content.

The scope of the present disclosure is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present disclosure and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present disclosure have been shown and described, it will be obvious to those skilled in the art that changes and modifications may be made without departing from the spirit and scope of the invention. The matter set forth in the foregoing description and accompanying drawings is offered by way of illustration only and not as a limitation.

1. A method for lysing bacterial cells, the method comprising:
(a) adding a lysis solution to a bacterial cell suspension to obtain a first mixture, shaking the first mixture at a rotation speed ranging from about 10 rpm to about 30 rpm, for about 1 minute to about 10 minutes, and incubating the first mixture to obtain a preliminary bacterial cell lysate; and
(b) adding a buffer to the preliminary bacterial cell lysate to obtain a second mixture, shaking the second mixture at a rotation speed ranging from about 10 rpm to about 70 rpm, for about 1 minute to about 10 minutes, and incubating the second mixture to obtain a bacterial cell lysate.

2. The method of claim 1, wherein in step (a), the first mixture is shaken at a rotation speed ranging from about 10 rpm to about 20 rpm.

3. The method of claim 1, wherein in step (a), the first mixture is shaken for about 1 minute to about 8 minutes.

4. The method of claim 1, wherein in step (a), the first mixture is shaken for about 3 minutes to about 8 minutes.

5. The method of claim 1, wherein in step (b), the second mixture is shaken at a rotation speed ranging from about 30 rpm to about 55 rpm.

6. The method of claim 1, wherein in step (b), the second mixture is shaken at a rotation speed ranging from about 40 rpm to about 50 rpm.

7. The method of claim 1, wherein in step (b), the second mixture is shaken for about 3 minutes to about 8 minutes.

8. The method of claim 1, wherein the lysis solution is an alkaline lysis buffer.

9. The method of claim 8, wherein the alkaline lysis buffer comprises 1% SDS (w/v) and 200 mM NaOH.

10. The method of claim 1, wherein the buffer is an acetate buffer.

11. The method of claim 10, wherein the acetate buffer is a potassium acetate buffer.

12. A method for preparing a plasmid, the method comprising:
(a) lysing bacterial cells by the method of claim 1 to obtain a bacterial cell lysate; and
(b) isolating the plasmid from the bacterial cell lysate.
